(19) United States
(12) Patent Application Publication
(10) Pub. No.: US 2008/0293980 A1
(43) Pub. Date: Nov. 27, 2008

(54) Method for the Synthesis of Aromatic Hydrocarbons From C1-C4-Alkanes and Utilization of C1-C4-Alkane-Comprising Product Stream

(76) Inventors: Frank Kiesslich, Dietzenbach (DE); Sven Crone, Limburgerhof (DE); Otto Machhammer, Mannheim (DE); Frederik van Laar, Dubai (AE); Ekkehard Schwab, Neustadt (DE); Gotz-Peter Schindler, Ludwigshafen (DE)

Correspondence Address: CONNOLLY BOVE LODGE & HUTZ LLP, 1875 EYE STREET, N.W., SUITE 1100, WASHINGTON, DC, 20006, US

(21) Appl. No.: 12/091,874

(22) PCT Filed: Oct. 30, 2006
(86) PCT No.: PCT/EP06/67938

§ 371 Date: Apr. 28, 2008

(30) Foreign Application Priority Data
Oct. 28, 2005 (DE) 10 2005 052 094.4
Apr. 27, 2006 (EP) 06113231..2

(51) Int. Cl. C07C 15/00

(52) U.S. Cl. 585/408; 585/415

(57) ABSTRACT

The present invention relates to a method for producing an aromatic hydrocarbon from a C₁-C₄-alkane, or a mixture of C₁-C₄-alkanes, which comprises
a) bringing a feedstock stream A which comprises a C₁-C₄-alkane, or a mixture of C₁-C₄-alkanes, into contact with a catalyst and reacting a part of the C₁-C₄-alkane, or a part of the mixture of the C₁-C₄-alkanes, to form aromatic hydrocarbon(s);
b) fractionating the product stream B resulting from step a) into a low-boiler stream C which comprises the majority of the hydrogen and of the unreacted C₁-C₄-alkane, or of the mixture of C₁-C₄-alkanes, and a high-boiler stream D, or a plurality of high-boiler streams D′, which stream or streams comprises or comprise the majority of the aromatic hydrocarbon formed; and
c) feeding the low-boiler stream C to a further C₁-C₄-alkane-consuming method, if appropriate the hydrogen present in the low-boiler stream C being separated off in advance.

Method for the synthesis of aromatic hydrocarbons from C₁-C₄-alkanes and utilization of C₁-C₄-alkane-comprising product stream

The present invention relates to a method for producing aromatic hydrocarbons such as benzene, toluene, ethylbenzene, styrene, xylene, naphthalene, or mixtures thereof, from C₁-C₄-alkanes, and the utilization of unreacted C₁-C₄-alkanes in a further C₁-C₄-alkane-consuming, in particular methane-consuming, method.

Aromatic hydrocarbons, such as benzene, toluene, ethylbenzene, styrene, xylene and naphthalene, are important intermediates in the chemical industry, the requirement for which continues to increase. Generally they are obtained from naphthalene by catalytic reformation, which naphthalene itself is obtained from mineral oil. Recent studies show that the worldwide stocks of mineral oil, compared with stocks of natural gas, are more limited. Therefore, it is worth seeking to produce aromatic hydrocarbons from feedstocks which can be obtained from natural gas. The main components of natural gas are methane (typical composition of natural gas: 75 to 99 mol % methane, 0.01 to 15 mol % ethane, 0.01 to 10 mol % propane, up to 0.06 mol % butane and higher hydrocarbons, up to 0.30 mol % carbon dioxide, up to 0.30 mol % of hydrogen sulfide, up to 0.15 mol % nitrogen, up to 0.05 mol % helium).

Anderson et al. report in Applied Catalysis 19, 141 (1985) that it is possible to react methane with nitrogen oxide (N₂O) in the presence of the catalyst H-ZSM-5 to form benzene. In the subsequent period, Claridge et al. (Applied Catalysis, A: General 89, 103 (1992)) established that methane can be reacted with oxygen, in particular in the presence of chloride-promoted manganese(IV) oxide to form, inter alia, benzene.

In addition, Wang et al. in Catalytic Letters 21, 35 (1993) describe a method for the dehydrogenation and aromatization of methane under non-oxidative conditions. With the ZSM-5 zeolite catalysts used in this case, which are modified with molybdenum or zinc, a methane conversion rate of max. 7.2% is achieved. In addition Qi et al. in Catalysis Today 98, 639 (2004) report that the methane conversion rate and the benzene selectivity of the Mo/ZSM-5 zeolite catalyst can be increased, inter alia, by addition of copper as promoter.

The previously described methods have low conversion rates of methane. Therefore, in US 2003/0144565 it is proposed to recirculate the product stream from which the aromatic hydrocarbons which have been formed have been separated off and thus further utilize the unreacted methane. However, recirculation streams lower the economic efficiency, since, firstly, further processing operations, such as renewed heating, compression etc. must be carried out and in addition greater reactor volumes need to be provided. In addition, undesired minor components or even undesired side reactions can occur.

The purpose of the present invention is to provide a method for producing aromatic hydrocarbons from C₁-C₄-alkanes, the C₁-C₄-alkanes used being utilized efficiently.

A method has now been found for producing aromatic hydrocarbons such as benzene, toluene, ethylbenzene, styrene, xylene, naphthalene, or mixtures thereof, from C₁-C₄-alkanes, and the utilization of unreacted C₁-C₄-alkanes in a further C₁-C₄-alkane-consuming method.

In particular, the invention relates to a method for producing an aromatic hydrocarbon from a C₁-C₄-alkane, or a mixture of C₁-C₄-alkanes, which comprises
a) bringing a feedstock stream A which comprises a C₁-C₄-alkane, or a mixture of C₁-C₄-alkanes, into contact with a catalyst and reacting a part of the C₁-C₄-alkane, or a part of the mixture of the C₁-C₄-alkanes, to form aromatic hydrocarbon(s);
b) fractionating the product stream B resulting from step a) into a low-boiler stream C which comprises the majority of the hydrogen and of the unreacted C₁-C₄-alkane, or of the mixture of C₁-C₄-alkanes, and a high-boiler stream D, or a plurality of high-boiler streams D′, which streams comprise the majority of the aromatic hydrocarbon formed and further high-boiling components; and
c) feeding the low-boiler stream C to a further C₁-C₄-alkane-consuming method, if appropriate the hydrogen present in the low-boiler stream C being separated off in advance.

In an embodiment, the feedstock stream A comprises at least 50 mol %, preferably at least 60 mol %, particularly preferably at least 70 mol %, exceptionally preferably at least 80 mol %, in particular at least 90 mol % C₁-C₄-alkane.

In particular, as feedstock stream A, use can be made of gas which comprises a fraction of at least 70 mol % methane, preferably at least 75 mol % methane. Generally, the feedstock stream, in addition to methane, also comprises ethane, customarily 0.01 to 15 mol %, propane, customarily 0.01 to 10 mol %, if appropriate butane and higher hydrocarbons, customarily 0 to 0.06 mol. The fraction of aromatic hydrocarbons is generally less than 2 mol %, and preferably less than 0.5 mol %.

In a further embodiment, as feedstock stream A, use can be made of LPG (liquid petroleum gas).

In a further embodiment, as feedstock stream A, use can be made of LNP (liquefied natural gas).

In addition, the feedstock stream A can comprise nitrogen, customarily 0 to 0.15 mol %, hydrogen sulfide, customarily 0 to 0.30 mol %, and/or other impurities, customarily 0 to 0.30 mol %.

In a further embodiment, hydrogen, steam, carbon monoxide, carbon dioxide, nitrogen, one or more noble gases and/or an oxygen-comprising gas stream can be additionally added to the feedstock stream A. As oxygen-comprising gas streams, for example air, enriched air, pure oxygen, come into consideration.

In a particular embodiment, the feedstock stream A is used in pure form.

Depending on the selected procedure and reaction conditions, a very high concentration or great dilution of the C₁-C₄-alkanes in one of the abovementioned gas streams can be a great advantage. The volume ratio between the feedstock stream A and the added gas stream can, depending on method, vary within very wide limits. Typically, this is in the range from 1000:1 to 1:500, preferably 1000:1 to 1:100, particularly preferably, in particular 1000:1 to 1:50.

The addition here can proceed in the form of a continuous stream or in a nonsteady state or periodic manner.

In a special embodiment, the metering of individual components can also be performed in traces of only some ppm to the feedstock stream A.

The dehydrogenating aromatization of C₁-C₄-alkanes according to the present invention can be carried out with feed or without feed of oxygen-comprising gases, in the presence of known catalysts under conditions known to those skilled in the art.

Suitable catalysts are, in particular, zeolite-comprising catalysts. These zeolites generally have a pore radius between 5 and 7 Angström. Examples of these are ZSM-zeolites, such as, for example, ZSM-5, ZSM-8, ZSM-11, ZSM-23 and ZSM-35, preferably ZSM-5, or MCM-zeolites, such as, for example, MCM-22. The catalysts, in addition to the zeolites, can comprise one or more metals from groups IIA, IIIA, IB, IIB, IIIB, VIIB, VIIB and VIIIB.

Preferably, use is made of Mo/HZSM-5 catalysts, which can be promoted with Cu, Co, Fe, Pt, Ru. However, it is also possible to promote with Sr, La or Ca. However, it is also possible to make use of W/HZSM-5, In/HZSM-5, Ga/HZSM-5, Zn/HZSM-5, Re/HZSM-5, or else W/HZSM-5, promoted with Mn, Zn, Ga, Mo or Co.

Likewise, preferably, use is made of W/MCM-22 catalysts which can be promoted with Zn, Ga, Co, Mo. However, Re/HMCM-22 can also be used.

Likewise, use can be made of chloride-promoted manganese(IV) oxide, H-ZSM-5 and Cu-W/HZSM-5, or else Rh on an SiO₂ support.

In one embodiment, operations are carried out with feed of oxygen-comprising components. As oxidizing agent, use can be made of customary oxidizing agents known to those skilled in the art for gas-phase reactions, such as, for example, oxygen, enriched air or air. Alternatively, depending on availability, under some circumstances, other oxidizing agents such as, for example, nitrogen oxides (NOₓ, N₂O) can also be utilized. In this case the oxidizing agent can be combined upstream of the reactor with the feedstock stream A or it can be added at one or more points in the reaction zone. Single addition or else addition in a plurality of portions is conceivable.

A special embodiment is the autothermal procedure. An autothermal procedure is taken to mean, in endothermic reactions, generating the heat for the process from the reaction mixture itself. For this, the endothermic target reaction is thermally coupled to a second reaction which makes up the balance via its exothermy. Heat supplied to the reaction zone via an external heating medium from the exterior is prevented by this. Thermal integration within the processes, however, can still be utilized.

The autothermal procedure can proceed in the most varied manners known to those skilled in the art.

In this case a second reaction which proceeds exothermally is utilized in order to make up thermally for the endothermy of the dehydrogenating aromatization. Preferably, this exothermic reaction is an oxidation. Various oxidizing agents can be utilized in this case. Customarily, oxygen, oxygen-comprising mixtures, or air are used.

Generally, the amount of the oxygen-comprising gas stream added to the reaction gas mixture is selected in such a manner that by combustion of the hydrogen present in the reaction gas mixture and if appropriate of hydrocarbons present in the reaction gas mixture and/or of carbon present in the form of coke, the amount of heat required for the dehydrogenating aromatization is generated. Customarily, a ratio O:C atom (mol/mol) of 1:12 to 1:1, preferably 1:10 to 1:15, in particular 1:15 to 1:2 is used. As oxygen-comprising gas stream, use is made of an oxygen-comprising gas which comprises inert gases, for example air, or oxygen-enriched air, or oxygen.

The hydrogen burnt for heat generation is the hydrogen formed in the dehydrogenating aromatization and also if appropriate hydrogen additionally added to the reaction gas mixture as hydrogen-comprising gas. Preferably, as much hydrogen should be present so that the molar ratio H₂/O₂ in the reaction gas mixture immediately after the feed of the oxygen-comprising gas is 1 to 10, preferably 2 to 5 mol/mol. In the case of multistage reactors, this applies to each intermediate feed of oxygen-comprising and, if appropriate, hydrogen-comprising, gas.

In an embodiment, operations are carried out in the presence of one or more oxidation catalysts which selectively catalyze the combustion of hydrogen with oxygen to form water in the presence of hydrocarbons. Combustion of these hydrocarbons with oxygen to form CO, CO₂ and water proceeds thereby only to a minor extent. Preferably, the dehydrogenating aromatization catalyst and the oxidation catalyst are present in different reaction zones.

In the case of multistage reaction procedure, the oxidation catalyst can be present in only one, in a plurality, or in all, reaction zones.

Preferably, the oxidation catalyst which selectively catalyzes the oxidation of hydrogen is arranged at the points at which higher oxygen partial pressures prevail than at other points of the reactor, in particular in the vicinity of the feed point for the oxygen-comprising gas stream. The feed of oxygen-comprising gas stream and/or hydrogen-comprising gas stream can proceed at one or more points of the reactor.

In a special embodiment of the inventive method, an intermediate feed of oxygen-comprising gas stream and, if appropriate, hydrogen-comprising gas stream proceeds upstream of each stage of a staged reactor. In a further embodiment of the inventive method, oxygen-comprising gas stream and if appropriate hydrogen-comprising gas stream is fed in upstream of each stage apart from the first stage. In an embodiment, downstream of each feed point a layer of a special oxidation catalyst is present, followed by a layer of the dehydrogenating aromatization catalyst. In a further embodiment, no special oxidation catalyst is present.

A preferred oxidation catalyst which selectively catalyzes the combustion of hydrogen comprises oxides and/or phosphates, selected from the group consisting of the oxides and/or phosphates of germanium, tin, lead, arsenic, antimony or bismuth. A further preferred catalyst which catalyzes the combustion of hydrogen comprises a noble metal of subgroup VIII. and/or I.

Depending on embodiment of the autothermal dehydrogenating aromatization of C₁-C₄-alkanes, the oxygen-comprising gas stream can be passed into the reaction zone together with, or separately from, the feedstock stream A. Analogous conditions also apply to the hydrogen-comprising gas stream.

The reactors are generally fixed-bed or fluid-bed reactors.

The temperature required in the dehydrogenation and aromatization of the C₁-C₄-alkane, preferably methane, can be achieved by heating. However, it is also possible to react a part of the feedstock stream A, preferably the methane, or the unreacted C₁-C₄-alkane and/or the hydrogen formed (which if appropriate has been recirculated) with oxygen, so that the reaction can be carried out autothermally. This can proceed, for example, according to methods known to those skilled in the art, for example in a two-zone reactor. In this case, in the first zone the oxygen is reacted and the energy liberated is used to heat feedstock stream A; in the second zone, the dehydrogenation and aromatization takes place. However, it is also possible to carry out the reaction of the oxygen with energy liberation and the dehydrogenation and aromatization in parallel.

The abovementioned reaction with oxygen can proceed in the form of a homogeneous gas-phase reaction, a flame, in a burner or in the presence of a contact catalyst.

For example, the hydrocarbon-comprising feedstock stream is combined with an oxygen-comprising stream and the oxygen is reacted in an oxidation reaction.

Suitable catalysts for the dehydrogenating aromatization with addition of oxygen are, in particular, the metal oxides described by Claridge et al. (Applied Catalysis, A: General: 89, 103 (1992)), in particular chloride-promoted manganese(IV) oxide. The reaction is accordingly customarily carried out at a temperature of 800 to 1100° C., preferably at 900 to 1100° C., in a pressure range from 1 to 25 bar, preferably 3 to 20 bar. The molar ratio of C₁-C₄-alkane, in particular methane, to oxygen is generally 30:1 to 5:1.

In addition, the catalyst H-ZSM-5 used by Anderson (Applied Catalysis 19, 141 (1985)), can also be used, in particular in the presence of nitrogen oxide. The reaction is customarily carried out at a temperature of 250 to 700° C., in particular at 300 to 600° C., in a pressure range of 1 to 10 bar. The molar ratio of C₁-C₄-alkane, in particular methane, to nitrogen oxide is generally 80:20 to 95:5.

In a further embodiment, the procedure is also operated without feed of oxygen-comprising gases.

Suitable catalysts are, in particular, zeolite-comprising, in particular ZSM-zeolites, such as, for example, ZSM-5, ZSM-8, ZSM-11, ZSM-23 and ZSM-35, preferably ZSM-5, or MCM-zeolites, such as, for example, MCM-22. The catalysts can, in addition to the zeolites, comprise one or more metals from groups IIIA, IB, IIB, VIIB, VIIB and VIIIB. Preferably, use is made of Mo/HZSM-5 catalysts, which can be promoted with Cu, Co, Fe, Pt, Ru. However, use can also be made of W/HZSM-5, In/HZSM-5, Ga/HZSM-5, Zn/HZSM-5, Re/HZSM-5, but also W/HZSM-5, promoted with Mn, Zn, Ga, Mo or Co. Likewise preferably, use is made of W/MCM-22 catalysts, which can be promoted with Zn, Ga, Co, Mo. However, Re/HMCM-22 can also be used.

In particular, use can be made of aluminosilicates of the pentasil type, for example ZSM-5, ZSM-8, ZSM-11, ZSM-23 and ZSM-35, preferably ZSM-5. These can be modified with molybdenum, zinc, gallium, preferably molybdenum. It is also possible to add a further metal, for example copper, or else cobalt, iron, platinum or ruthenium. Thus, for example, copper-promoted Mo/ZSM-5 zeolite catalysts are obtained (Qi et al., Catalysis Today 98, 639 (2004)), or Ga/ZSM-5 zeolite catalysts promoted by a metal of group VIIIB of the periodic table of the elements, in particular by rhenium (U.S. Pat. No. 4,727,206) are obtained. Particular molybdenum-modified ZSM-5 zeolites are described in WO 02/10099.

In addition, use can also be made of MCM/22 catalysts which are modified by W and promoted, if appropriate, by Zn, Ga, Co, Mo. Likewise suitable is Re/HMCM-22.

Customarily, the dehydrogenating aromatization of C₁-C₄-methane which proceeds without supply of oxygen-comprising gas streams is carried out in the presence of the abovementioned catalysts at temperatures of 400 to 1000° C., preferably from 500 to 900° C., particularly preferably from 600 to 800° C., in particular from 700 to 750° C., at a pressure of 0.5 to 100 bar, preferably at 1 to 50 bar, particularly preferably at 1 to 30 bar, in particular 1 to 10 bar. Customarily, the reaction is carried out at a GHSV (Gas Hourly Space Velocity) of from 100 to 10 000 h⁻¹, preferably from 200 to 3000 h⁻¹.

It can be advantageous to activate, before actual use, the catalysts used in the dehydrogenating aromatization of C₁-C₄-alkanes which is operated without supply of oxygen-comprising gas streams.

This activation can be performed using a C₂-C₄-alkane, such as, for example, ethane, propane, butane or a mixture thereof, preferably butane. The activation is carried out at a temperature of from 250 to 650° C., preferably at 350 to 550° C., and at a pressure of 0.5 to 5 bar, preferably at 0.5 to 2 bar. Customarily, the GHSV (Gas Hourly Space Velocity) in the activation is 100 to 4000 h⁻¹, preferably 500 to 2000 h⁻¹.

However, it is also possible to carry out an activation by the feedstock stream A already comprising per se the C₂-C₄-alkane, or a mixture thereof, or adding the C₂-C₄-alkane, or a mixture thereof, to the feedstock stream A. The activation is carried out at a temperature of from 250 to 650° C., preferably at 350 to 550° C., and at a pressure of 0.5 to 5 bar, preferably at 0.5 to 2 bar. Customarily, the GHSV (Gas Hourly Space Velocity) in the activation is 100 to 4000 h⁻¹, preferably 500 to 2000 h⁻¹.

In a further embodiment, it is also possible additionally to add hydrogen to the C₂-C₄-alkane.

Of course, the catalysts used in this method, in particular when the method is carried out without addition of oxygen-comprising gas streams, when their activity decreases, can be regenerated by customary methods known to those skilled in the art. Here, mention may be made of, in particular, the treatment with an oxygen-comprising gas stream, such as, for example air, enriched air or pure oxygen, by passing the oxygen-comprising gas stream instead of the feedstock stream A, over the catalyst. However, it is also possible to regenerate the catalysts using hydrogen. This can be performed by adding, for example, a hydrogen stream to the feedstock stream A. The ratio of hydrogen stream to feedstock stream A is customarily in the range from 1:1000 to 2:1, preferably 1:500 to 1:5. However, it can also be advisable to pass feedstock stream A and hydrogen alternately over the catalyst.

The feedstock stream A is brought into contact with the catalyst in a reaction zone, the reaction zone, inter alia, being able to be represented by a reactor, a plurality of series-connected reactors, or one or more reactors in cascade.

The dehydrogenating aromatization can be carried out in principle in all reactor types from the prior art. A comparatively extensive description of inventively suitable reactor types is also contained in “Catalytica® Studies Division, Oxidative Dehydrogenation and Alternative Dehydrogenation Processes” (Study Number 4192 OD, 1993, 430 Ferguson Drive, Mountain View, Calif., 9404-35272, USA).

A suitable reactor form is the fixed-bed tubular reactor or tube-bundle reactor. In the case of these, the catalyst is situated as a fixed bed in a reaction tube or in a bundle of reaction tubes. Customary reaction tube internal diameters are about 10 to 15 cm. A typical dehydrogenating aromatization tube-bundle reactor comprises approximately 300 to 1000 reaction tubes. The catalyst geometry can be, for example, bead-shaped or cylindrical (hollow or solid), ring-shaped, saddle-shaped or tablet-shaped. In addition, extrudates, for example in extruded rod, trilobe, quadrulobe, star or hollow cylinder shape come into consideration.

The dehydrogenating aromatization can also be catalyzed heterogeneously in the fluidized bed. In a particular embodiment, the reactor comprises a fluidized bed, but it can also be expedient to operate a plurality of fluidized beds next to each other, of which one or more generally finds itself in the regeneration or reactivation state. The heat required for the dehydrogenating aromatization can be introduced in this case into the reaction system by preheating the catalyst to the reaction temperature. By admixing an oxygen-comprising gas stream, the preheater can be dispensed with, and the required heat is generated directly in the reactor system by combustion of hydrogen and/or hydrocarbons in the presence of oxygen (autothermal procedure).

The dehydrogenating aromatization can be carried out in a staged reactor. This comprises one or more sequentially following catalyst beds. The number of the catalyst beds can be 1 to 20, expediently 1 to 6, preferably 1 to 4, and in particular 1 to 3. Reaction gas flows through the catalyst beds preferably radially or axially. Generally, such a staged reactor is operated using a fixed-bed catalyst. In the simplest case, the fixed-bed catalysts are arranged axially in a shaft furnace reactor, or in the ring gaps of concentrically arranged cylindrical gratings. A shaft furnace reactor corresponds to a staged reactor having only one stage. Carrying out the dehydrogenating aromatization in a single shaft furnace reactor corresponds to one embodiment. In a further preferred embodiment, the dehydrogenating aromatization is carried out in a staged reactor having 3 catalyst beds.

Product stream B preferably comprises one or more aromatic hydrocarbons selected from the group benzene, toluene, ethylbenzene, styrene, xylene and naphthalene. In particular, product stream B comprises, as aromatic hydrocarbon, benzene, naphthalene or mixtures thereof, particularly preferably benzene, likewise particularly preferably benzene and naphthalene.

The yield of aromatic hydrocarbon(s) (based on reacted alkane from feedstock stream A) is in the range from 1 to 95%, preferably from 5 to 80%, more preferably from 10 to 60%, particularly preferably from 15 to 40%.

The selectivity for aromatic hydrocarbon(s) (based on reacted alkane from feedstock stream A) is at least 10%, preferably 30%, particularly preferably 50%, exceptionally preferably 70%, in particular 90%.

In addition, product stream B, in addition to unreacted C₁-C₄-alkane, or a mixture of unreacted C₁-C₄-alkanes and hydrogen formed, comprises inert substances already present in feedstock stream A such as nitrogen, helium (and if appropriate alkanes such as ethane, propane etc.) and also byproducts formed and other impurities already present in feedstock stream A and also if appropriate (in part) gas streams added to feedstock stream A.

If operated autothermally, the product stream can additionally comprise the water, carbon monoxide and/or carbon dioxide formed in the reaction with oxygen.

In a special embodiment, partial recycling of product stream B can be carried out, that is before separating off high-boilers. For this, a part of the product stream B coming from the reaction zone is recirculated to the reaction zone. This can be performed optionally by direct metering into the reaction zone or by prior combination with feedstock stream A. The fraction of the recycled stream is between 1 and 95% of product stream B, preferably between 5 and 90% of product stream B.

Alternatively to the above recycling, in a further embodiment, recycling a part of the low-boiler streams C and C′ can also be performed. These low-boiler streams C and C′ are obtained by partially or completely separating off the high-boilers and the aromatic hydrocarbons from the product stream B. A part of the streams C and/or C′ are optionally recirculated by direct metering into the reaction zone or by prior combination with feedstock stream A. The fraction of the recirculated stream is between 1 and 95% of the corresponding stream C or C′, preferably between 5 and 90% of the corresponding stream C or C′.

If appropriate, the recycled streams can be wholly or partly freed from hydrogen.

The recycling of a stream can be performed, for example, using a compressor, a fan or a nozzle. In a preferred embodiment, the nozzle is a propulsive jet nozzle, feedstock stream A or an oxygen-comprising stream or a vapor stream being used as propulsive medium.

The product stream B is separated into the low-boiler stream C and the high-boiler stream D by condensation or else fractional condensation. Fractional condensation is here taken to mean a multistage distillation in the presence of relatively large amounts of inert gas. For instance, product stream B can be cooled to −30° C. to 80° C., preferably to 0° C. to 70° C., particularly preferably to 30° C. to 60° C. In this case the aromatic hydrocarbons and high-boilers condense, whereas the unreacted methane and the hydrogen formed are present in the gaseous state and thus cannot be separated off by conventional methods. If appropriate, the low-boiler stream C also comprises the abovementioned inert substances and alkanes and also the byproducts formed and/or impurities already present in feedstock stream A and also if appropriate (in part) gas streams added to feedstock stream A (Fig. I).

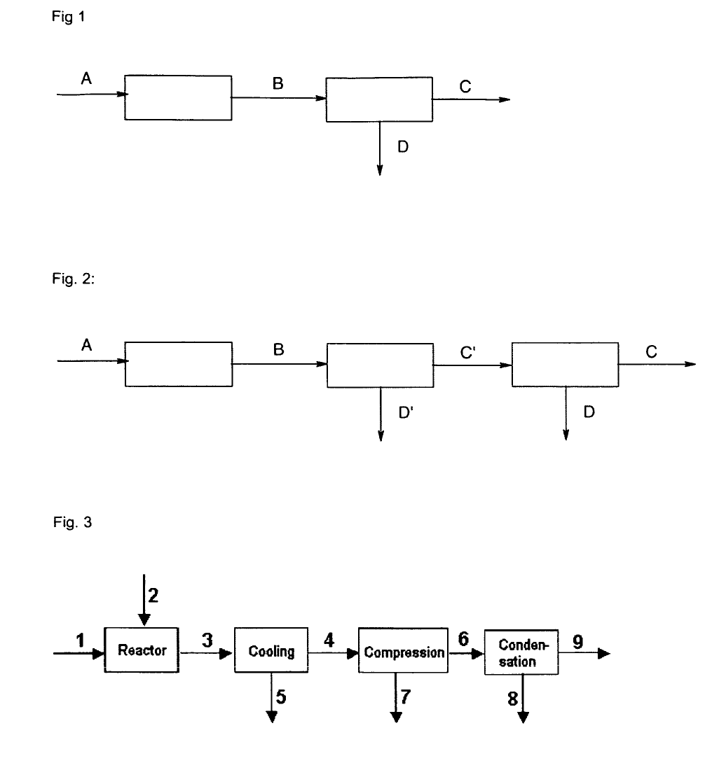

In a particular embodiment, it can be advantageous to free the product stream B from high-boilers in a plurality of stages. For this, it is cooled, for example to −30° C. to 80° C., the high-boiler stream D′ which comprises a part of the high-boilers is separated off and the low-boiler stream C′ is compressed and further cooled so that the high-boiler stream D and the low-boiler stream C are obtained. Compression is performed, preferably to a pressure level of 5 to 100 bar, more preferably 10 to 75 bar, and further preferably 15 to 50 bar. To achieve substantial condensation of a defined compound, a correspondingly suitable temperature is set. If the condensation proceeds below 0° C., if appropriate, prior drying of the gas is necessary. (FIG. 2)

The high-boiler stream D principally comprises the lighter aromatic hydrocarbons, such as, for example benzene, and the low-boiler stream C comprises the unreacted C₁-C₄-alkanes, preferably methane, the hydrogen formed and if appropriate the abovementioned inert substances and also the highly volatile byproducts formed and/or impurities already present in feedstock stream A. The unreacted C₁-C₄-alkane, preferably methane, and the hydrogen formed can be separated if desired by customary methods.

In addition, the low-boiler stream C, in the case of the autothermal procedure, can comprise the carbon monoxide, carbon dioxide formed in the reaction with oxygen.

The aromatic hydrocarbons present in the high-boiler stream D can be separated and/or purified by customary methods. If appropriate, the high-boiler stream D, in the case of the autothermal procedure, can comprise the water formed in the reaction with oxygen, which can be separated off in a customary manner, for example via a phase separator.

Depending on procedure of the dehydrogenating aromatization (with addition of oxygen or without addition of oxygen) and the requirements of the continuing process with respect to purity, the presence of interfering components or the calorific value of the low-boiler stream C, it can be necessary to separate off individual by components before further use.

For instance, in the case of dehydrogenating aromatization with addition of oxygen, under some circumstances carbon monoxide or carbon dioxide can be formed for example. When atmospheric oxygen is used, the nitrogen or other inert materials co-introduced into the system can be present in stream C.

The dehydrogenating aromatization is associated with the formation of hydrogen, as a result of which the calorific value of the low-boiler stream C changes.

In a special embodiment of the present invention, therefore, one or more of these components formed which are not C₁-C₄-alkanes, are in part or completely separated off.

For this, for example in a method part between separating off the high-boilers (D, D′) and the further continuing process, the non-condensable or low-boiling gas constituents such as hydrogen, oxygen, carbon monoxide, carbon dioxide, nitrogen are separated off from the hydrocarbons in an absorption/desorption cycle by means of a high-boiling absorption medium, a stream being obtained which comprises the C₁-C₄-hydrocarbons and the absorption medium, and an exhaust gas stream which comprises the non-condensable or low-boiling gas components.

Inert absorption media used in the absorption stage are generally high-boiling nonpolar solvents in which the C₁-C₄-hydrocarbon mixture to be separated off has a significantly higher solubility than the remaining gas components to be separated off. The absorption can proceed by simply passing through the stream C through the absorption medium. However, it can also proceed in columns. In this case, cocurrent flow, countercurrent flow or cross current flow can be employed. Suitable absorption columns are, for example, tray columns having bubble-cap trays, valve trays and/or sieve trays, columns having structured packings, for example cloth packings or metal sheet packings having a specific surface area of from 100 to 1000 m²/m³ such as Mellapak® 250 Y, and random packing columns, for example having beads, rings or saddles made of metal, plastic or ceramic as random packings. However, trickling and spray towers, graphite block absorbers, surface absorbers such as thick-layer and thin-layer absorbers, and also rotary columns, disk scrubbers, cross flow mist scrubbers, rotary scrubbers and bubble columns with and without internals also come into consideration.

Suitable absorption media are relatively nonpolar organic solvents, for example aliphatic C₅-C₁₈-alkenes, naphtha or aromatic hydrocarbons such as the middle oil fractions from paraffin distillation, or ethers having bulky groups, or mixtures of these solvents, a polar solvent such as 1,2-dimethyl phthalate being able to be added to these. Suitable absorption media are, in addition, esters of benzoic acid and phthalic acid with straight-chain C₁-C₈-alkanols, such as n-butyl benzoate, methyl benzoate, ethyl benzoate, dimethyl phthalate, diethyl phthalate, and also abovementioned heat carrier oils, such as biphenyl and diphenyl ether, their chlorine derivatives, and also triaryl alkenes. A suitable absorption medium is a mixture of biphenyl and diphenyl ether, preferably in the azeotropic composition, for example the commercially available Diphyl®. Frequently, this solvent mixture comprises dimethyl phthalate in an amount of 0.1 to 25% by weight. Suitable absorption media are, in addition, pentanes, hexanes, heptanes, octanes, nonanes, decanes, undecanes, dodecanes, tridecanes, tetradecanes, pentadecanes, hexadecanes, heptadecanes and octadecanes, or fractions isolated from refinery streams which comprise said linear alkanes as main components.

Alternatively, carbon dioxide can also be removed from stream C in a targeted manner using a selective absorption medium. In turn, absorption media, such as, for example, basic scrubbing media know to those skilled in the art in which the carbon dioxide to be separated off has a markedly higher solubility than the remaining gas components to be separated off can be used in the absorption stage. The absorption can be performed by simply passing stream C through the absorption medium. However, it can also proceed in columns. The procedure can be carried out in cocurrent flow, countercurrent flow or cross current flow. Technically, in this case, the apparatus solutions set forth above come into consideration.

For separating off the hydrogen present in the exhaust gas stream it can be passed, if appropriate after cooling has been performed, for example in an indirect heat exchanger, through a membrane generally constructed as a tube, which is only permeable to molecular hydrogen.

Alternatively, individual components can also be separated off by chemical reaction. By oxidation of the resultant hydrogen, for example, it may be removed as water from the mixture by condensation.

Alternatively, components can be separated off in an adsorption process (thermal or pressure-swing adsorption). In this case an adsorbent is charged in a cyclic manner in a first phase with the hydrogen-comprising stream, all components apart from hydrogen, thus also including the C₁-C₄-alkanes, being retained by adsorption. In a second phase, these components are desorbed again by lowered pressure or elevated temperature.

The molecular hydrogen thus separated off can, if required, be used at least in part in a hydrogenation, or else fed to another use, for example for generating electrical energy in fuel cells. Alternatively, the exhaust gas stream can be burnt.

Alternatively, if boiling points are sufficiently different, use can also be made of rectification for separating off individual components.

Separation of individual components is generally not quite complete, so that, in the C₁-C₄-hydrocarbons, depending on the type of separation, small amounts or else only traces of the further gas constituents can still be present.

The unreacted C₁-C₄-alkane present in the low-boiler stream C and the hydrogen formed can then be fed to a further C₁-C₄-alkane-consuming process. Examples of methane-consuming processes are
i) combustion in combined heat and power stations (with production of energy, heat and/or steam), in particular in combined-cycle power stations;
ii) making synthesis gas via steam reformers or partial oxidation;
iii) reaction with ammonia to give prussic acid in the presence of oxygen by the Andrussow method, or without addition of oxygen by the BMA method;
iv) reaction with sulfur to give carbon disulfide;
v) pyrolysis to give acetylene in electric arc, or by the Sachsse-Bartholome method;
vi) oxidative coupling to give ethylene.

As C₂-C₄-alkane-consuming processes, mention may be made of i) and ii).

It can be advantageous to separate off partially or completely the hydrogen formed before use in the methane-consuming process such as, for example, processes ii) to vi). For this, use can be made of the methods listed above which are known to those skilled in the art and the hydrogen thus produced can itself be used for energy production or in a hydrogen-consuming process, such as, for example, hydrogenation. It can likewise also be advantageous to separate off, using current methods, before use in the methane-consuming process, the abovementioned inert substances, alkanes and also the byproducts formed and the carbon dioxide formed in the case of the autothermal procedure.

Preferably, the low-boiler stream C is fed for combustion in combined heat and power stations for production of energy, heat and/or steam. Power stations for electricity generation having the highest efficiencies currently comprise modern combined cycle power stations (GuD power stations) which achieve efficiencies of about 50 to 60%.

A GuD power station is a heat engine whose actual efficiency depends, via the Carnot, the highest theoretically possible efficiency of a heat engine, on the temperature difference between heat source and heat sink. The heat source in a GuD power station corresponds to the combustion process, the heat sink to the ambient temperature or the cooling water. The theoretical relation between the efficiency E of a heat engine and the temperature difference is ε=1−(T_{S}/T_{Q}), wherein T_{S} is the temperature of the heat sink and T_{Q} is the temperature of the heat source, in each case stated in K. The efficiency of a heat engine is accordingly higher, the greater is the temperature difference between T_{S} and T_{Q}. For a GuD power station this means that, for the same expenditure for cooling (and therefore the same temperature of the heat sink), the efficiency is higher, the higher is the temperature of the combustion process.

In this case, surprisingly, in addition to the advantages already listed above of the method according to the invention, a further advantage is found especially of this embodiment of the method according to the invention, in which the low-boiler stream C is fed, in step c) of the method according to the invention, to a GuD power station. The combustion temperatures achieved with the low-boiler stream C in the GuD power station are significantly above those of a conventional methane combustion mix (natural gas), as can be seen in example 3. The efficiency of the GuD power station can therefore be increased and a high total efficiency of the overall process achieved.

In addition to the increased efficiency of the overall process, this embodiment of the invention has a further advantage: its CO₂ eco balance which is favorable in the overall process. As a result of the carbon utilization in the first stage in a chemical product, the H:C ratio in the gas resulting from the first stage (after separating off the aromatics) is higher. In the second process stage, therefore, less carbon needs to be burnt. This gas, for the same calorific value, therefore leads to lower CO₂ emissions.

In a further embodiment of the invention, use is made of the low-boiler stream C in stage c) of the method according to the invention as synthesis gas in ammonia synthesis. Compared with the usually conventional use of natural gas for generating the hydrogen required for ammonia synthesis, the use according to the invention of the low-boiler stream C for synthesizing the hydrogen required for ammonia synthesis exhibits a significantly lower requirement for natural gas for heating the process and also a markedly lower mass flow rate. This means that in the case of the use according to the invention of the low-boiler stream C for forming synthesis gas for ammonia production, significantly less methane per ton of ammonia needs to be used than is the case when natural gas is used for synthesis gas production. In particular, the advantages of this embodiment are exhibited on consideration of the overall process. Two commercially important products are produced, aromatics and ammonia, wherein by the combination of the dehydrogenating aromatization and ammonia synthesis, the second process can be carried out under more advantageous conditions than in the case of direct use of C₁-C₄-alkane-containing gas in ammonia synthesis.

According to the inventive method, a selectivity for aromatic hydrocarbon(s) (based on reacted alkane from feedstock stream A) is in the range of at least 10%, preferably 30%, particularly preferably 50%, exceptionally preferably 70%, in particular 90%.

### EXAMPLE 1

### Coupling to a Combined-Cycle Power Station (FIG. 3, Table 1)

Hereinafter, an inventive embodiment is simulated by computer, the plant having been designed for 100 kt/yr of benzene and 20 kt/yr of naphthalene. The dehydrogenating aromatization proceeds with a selectivity of 71% with respect to benzene, 14% with respect to naphthalene and 15% with respect to CO/CO₂. The conversion rate of methane is 23%.

Methane is expanded from approximately 50 bar to 1.2 bar. After preheating to 500° C., methane (stream 1) is fed to the reactor at a pressure of 1.2 bar. In addition, oxygen (stream 2) is fed for in-situ production of the required heat of reaction. Stream 3 (product stream B) leaves the reactor at 750° C. Stream 3 (product stream B) is cooled. Condensate 5 (heavy-boiler stream D′) formed predominantly comprises naphthalene and water, but can also small amounts of benzene and can be worked up accordingly. Gas stream 4 (low-boiler stream C′) is compressed in a multistage manner to 30 bar. In the intermediate cooling stages further condensate (stream 7) is produced which essentially comprises water. Subsequently stream 6 which has a temperature of 138° C. is partially condensed. Benzene and in turn water are separated off at a pressure of 30 bar. The unreacted methane and also the hydrogen formed and low-boiling byproducts are fed to the power station as stream 9.

TABLE 1

| Stream No. | 1 | 2 | 3 | 4 | 5 |
|---|---|---|---|---|---|
| Rate [kg/h] | 96 974 | 17 914 | 114 887 | 108 761 | 6126 |
| C₆H₁₀ | 0.0 | 0.0 | 2.27 | 0.1 | 40.81 |
| C₆H₈ | 0.0 | 0.0 | 11.25 | 11.88 | 0.06 |
| H₂O | 0.0 | 0.0 | 12.03 | 9.38 | 59.09 |
| CH₄ | 100 | 0.0 | 64.75 | 68.40 | 0.0 |
| CO | 0.0 | 0.0 | 1.72 | 1.82 | 0.0 |
| CO₂ | 0.0 | 0.0 | 5.4 | 5.71 | 0.04 |
| H₂ | 0.0 | 0.0 | 2.58 | 2.73 | 0.0 |
| O₂ | 0.0 | 100.0 | 0.0 | 0.0 | 0.0 |
| T [° C.] | 500 | 100 | 750 | 50 | 50 |
| p [bar] | 1.5 | 1.5 | 1.5 | 1.4 | 1.4 |

| Stream No. | 6 | 7 | 8 | 9 |
|---|---|---|---|---|
| Rate [kg/h] | 99 901 | 8860 | 13 989 | 85 912 |
| C₆H₁₀ | 0.0 | 1.2 | 0.0 | 0.0 |
| C₆H₈ | 12.92 | 0.17 | 88.00 | 0.7 |
| H₂O | 1.51 | 98.09 | 10.60 | 0.03 |
| CH₄ | 74.46 | 0.0 | 0.0 | 86.59 |
| CO | 1.98 | 0.0 | 0.0 | 2.3 |
| CO₂ | 6.16 | 0.54 | 1.4 | 6.94 |
| H₂ | 2.97 | 0.0 | 0.0 | 3.45 |
| O₂ | 0.0 | 0.0 | 0.0 | 0.0 |
| T [° C.] | 138 | 55 | 1 | 1 |
| p [bar] | 30 | 12.3 | 30 | 30 |

The figures in columns 1 to 9 with respect to C₆H₁₀, C₆H₆, H₂O, CO, CO₂, H₂ and O₂ are % by weight.

This example clearly shows that by using the inventive method, by operation in the straight-through procedure, at the preset space velocity, corresponding conversion rates can be achieved, and the volumes of the apparatuses required can be dimensioned to be correspondingly smaller and the mass streams are correspondingly smaller. Therefore, not only the procurement costs but also the operating costs in the inventive method are low.

### EXAMPLE 2

As catalyst, use was made of a Mo-/H-ZSM-5 catalyst (3% by weight Mo, Si:Al ratio of approximately 50 mol/mol). This was impregnated in a single-stage impregnation using an aqueous solution of ammonium heptamolybdate, dried and calcined at 500° C.

Approximately 1 g of the pulverulent catalyst was heated to 500° C. under helium. At this temperature methane was added and the catalyst was heated stepwise to 750° C. under helium/methane (approximately 15% helium in methane). Then, at this temperature, under the external atmosphere (approximately 1 bar, pressure drop over approximately 1 bar), the dehydrogenating aromatization was studied at a GHSV of approximately 1000 h⁻¹ (Table 2).

TABLE 2

| Methane | Helium | O₂ | CO₂ | CO | H₂O | H₂ | Ethylene | Benzene | Toluene | Naphthalene |
|---|---|---|---|---|---|---|---|---|---|---|
| [Vol %] | [Vol %] | [Vol %] | [Vol %] | [Vol %] | [Vol %] | [Vol %] | [Vol %] | [Vol %] | [Vol %] | [Vol %] |
| 60.87 | 16.69 | 0.00 | 0.00 | 0.15 | 0.00 | 11.15 | 0.00 | 1.21 | 0.05 | 0.04 |

### EXAMPLE 3

Subsequently the combustion of a gas mixture (a) containing C₁-C₄-alkane (natural gas, not according to the invention) which is conventionally used in GuD power stations, and also the combustion of a low-boiler fraction C (b) (according to the invention, exhaust gas DHAM) were calculated theoretically.

a) C₁-C₄-Alkane-Comprising Gas Mixture (Natural Gas; not According to the Invention)

TABLE 3
Composition

| | |
|---|---|
| H₂ | 0.00% |
| CH₄ | 93.00% |
| C₂H₄ | 0.00% |
| C₂H₆ | 3.00% |
| C₃H₈ | 1.30% |
| C₄H₁₀ | 0.60% |
| CO₂ | 1.00% |
| N₂ | 1.10% |
| Specific CO₂ formation: | 28.5 m³CO₂/kJ |
| Theoretical combustion temperature: | 1944° C. |

b)Low-boiler fraction C (exhaust gas DHAM; according to the invention)

TABLE 4
Composition

| | |
|---|---|
| H₂ | 30.00% |
| CH₄ | 64.00% |
| C₂H₄ | 1.00% |
| C₂H₆ | 2.00% |
| C₃H₈ | 1.30% |
| C₄H₁₀ | 0.60% |
| CO₂ | 0.00% |
| N₂ | 1.10% |
| Specific CO₂ formation: | 25.4 m³CO₂/kJ |
| Theoretical combustion temperature: | 1966° C. |

### EXAMPLE 4

Subsequently the use of methane (not according to the invention) and also of the low-boiler stream C in ammonia synthesis was simulated. The calculations were carried out for an ammonia yield of 100 t/h.

In Table 5 the respective feed compositions and also the mass streams resulting from the simulation calculations are shown.

TABLE 5

| | Methane | Low-boiler stream C |
|---|---|---|
| | (not according | (according to |
| Feed | to the invention) | the invention) |
| Composition | 100% CH₄ | 22.6% H₂ + 77.4% CH₄ |
| H₂ demand in kmol/h | 8824 | 8824 |
| Required feed in kmol/h | 4412 | 4974 |
| Required feed in t/h | 70.6 | 63.8 |

The ratio of the volumetric flow rates when actual conditions are taken into account is: feed (low-boiler stream C)/feed (methane)=1.20, the ratio of the mass flow rates feed (low-boiler stream C)/feed (methane)=0.94. This means that when the low-boiler stream C is used, a volumetric flow rate higher by 20% is necessary, but a mass flow rate lower by 6% is necessary, and therefore a lower amount of methane is necessary than when pure methane is used. The ratio of the demand of natural gas for heating the process is: heating natural gas demand (low-boiler stream C)/heating natural gas demand (methane)=0.942. The heat demand in the production of synthesis gas is therefore significantly lower in the case of the use according to the invention of the low-boiler stream C in the ammonia synthesis than when methane is used as feedstock.

1-22. (canceled)

23. A method for producing an aromatic hydrocarbon from a C₁-C₄-alkane, or a mixture of C₁-C₄-alkanes comprising
a) bringing a feedstock stream A which comprises a C₁-C₄-alkane, or a mixture of C₁-C₄-alkanes, into contact with a catalyst and reacting a part of the C₁-C₄-alkane, or a part of the mixture of the C₁-C₄-alkanes, to form aromatic hydrocarbon(s);
b) fractionating the product stream B resulting from step a) into a low-boiler stream C which comprises the majority of the hydrogen and of the unreacted C₁-C₄-alkane or of the mixture of C₁-C₄-alkanes and a high-boiler stream D, or a plurality of high-boiler streams D′, which stream or streams comprises or comprise the majority of the aromatic hydrocarbon formed; and
c) feeding the low-boiler stream C to a further C₁-C₄-alkane-consuming method and optionally the hydrogen present in the low-boiler stream C being separated off in advance.

24. The method according to claim 23, wherein the feedstock stream A) contains at least 70 mol % of methane.

25. The method according to claim 23, wherein hydrogen, carbon monoxide, carbon dioxide, one or more noble gases and/or oxygen-comprising gas streams and optionally steam are added to the feedstock stream A.

26. The method according to claim 23, wherein the dehydrogenating aromatization of the C₁-C₄-alkane or of a mixture of C₁-C₄-alkanes, proceeds with feed of oxygen-comprising gas streams.

27. The method according to claim 23, wherein the dehydrogenating aromatization of the C₁-C₄-alkane, or of the mixture of C₁-C₄-alkanes, proceeds with feed of oxygen-comprising gas streams.

28. The method according to claim 23, wherein the dehydrogenating aromatization of the C₁-C₄-alkane, or of the mixture of C₁-C₄-alkanes, is conducted in the presence of a zeolite-comprising catalyst.

29. The method according to claim 28, wherein the catalyst is activated by treatment with a C₁-C₄-alkane, or a mixture thereof.

30. The method according to claim 23, wherein the dehydrogenating aromatization of the C₁-C₄-alkane, or of the mixture of C₁-C₄-alkane s is conducted at a temperature from 400 to 1000° C., and at a total pressure from 0.5 to 100 bar,

31. The method according to claim 23, wherein the method is conducted autothermally.

32. The method according to claim 23 comprising recirculating to the reaction zone a part of the product stream B before separating off the high boilers or aromatic hydrocarbons.

33. The method according to claim 23, comprising recirculating to the reaction zone a part of the product stream B after partially or completely separating off high boilers or aromatic hydrocarbons.

34. The method according to claim 23, wherein the aromatic hydrocarbon present in the product stream B is benzene, toluene, ethylbenzene, styrene, xylene, naphthalene, or a mixture thereof.

35. The method according to claim 34, wherein the aromatic hydrocarbon present in the product stream B is benzene.

36. The method according to claim 34, wherein the aromatic hydrocarbons present in the product stream B is benzene and naphthalene.

37. The method according to claim 23, wherein C₁-C₄-consuming method is selected from one or more methods of the following groups:
i) combustion in combined heat power stations;
ii) making synthesis gas via steam reformers or partial oxidation;
iii) reaction with ammonia to give prussic acid in the presence of oxygen by the Andrussow method, or without addition of oxygen by the BMA method;
iv) reaction with sulfur to give carbon disulfide;
v) pyrolysis to give acetylene in electric arc, or by the Sachsse-Bartholome method; and
vi) oxidative coupling to give ethylene.

38. The method according to claim 37, wherein the C₁-C₄-consuming methods is combustion in combined heat and power stations (i).

39. The method according to claim 23, wherein the low-boiler stream C, before its use in a continuing method, is partially or completely freed in a separation from one or more components which are not C₁-C₄-alkane, or a mixture of C₁-C₄-alkanes.

40. The method according to claim 39, wherein the separation is an adsorption absorption, membrane or rectification separation step, or separation by chemical reaction.

41. The method according to claim 23, wherein the hydrogen present in the low-boiler stream C is partially or completely separated off.

42. The method according to claim 23, wherein the C₁-C₄-consuming method in step c) is making synthesis gas for an ammonia synthesis.

43. The method according to claim 42, wherein the ammonia synthesis directly follows the making of synthesis gas from the low-boiler stream C.

44. The method according to claim 37, wherein the C₁-C₄-consuming method is combustion in a combined cycle gas turbine.
